(19) United States
(12) Patent Application Publication
(10) Pub. No.: US 2011/0275933 A1
(43) Pub. Date: Nov. 10, 2011

(54) MOTION COMPENSATION IN NUCLEAR IMAGING

(75) Inventors: Joyoni DEY, Somerville, MA (US); Michael A. King, Northboro, MA (US)

(73) Assignee: UNIVERSITY OF MASSACHUSETTS, Shrewsbury, MA (US)

(21) Appl. No.: 13/125,608

(22) PCT Filed: Oct. 22, 2009
(86) PCT No.: PCT/US09/61719

§ 371 Date: Jul. 13, 2011

Related U.S. Application Data
(60) Provisional application No. 61/107,523, filed on Oct. 22, 2008.

(51) Int. Cl. A61B 6/03; A61B 6/00

(52) U.S. Cl. 600/428; 600/436

(57) ABSTRACT

The invention relates to methods and systems for compensating for respiratory motion of individuals during nuclear imaging. In some embodiments, the methods include obtaining data representing a reference respiratory state for the individual and obtaining data that represents a plurality of respiratory states that correspond to at least a portion of a cycle of respiration of the individual; comparing each of the plurality of respiratory states to a subset of the reference state data to obtain a motion estimate of each of the plurality of respiratory states; compensating for respiratory motion in each of the plurality of respiratory states based on the motion estimates to obtain motion-compensated respiratory state data; and constructing an image using the motion-compensated respiratory state data.

### CROSS REFERENCE TO RELATED APPLICATION

This application claims the benefit of Provisional Application No. 61/107,523 filed on Oct. 22, 2008, the entire contents of which are hereby incorporated by reference.

### STATEMENT OF GOVERNMENT RIGHTS IN INVENTION

Funding for development of the subject matter described herein was provided in part by grant R01 EB001457 from the National Institute of Biomedical Imaging and Bioengineering. The United States Federal Government may retain selected rights in any invention derived from this subject matter.

### TECHNICAL FIELD

This disclosure relates to nuclear imaging systems, and in particular, to methods and systems for compensating for patient respiratory motion.

### BACKGROUND

In nuclear imaging, one injects radioactive material into the patient's body. As the material decays, gamma rays leave the body and travel in certain directions, each of which is defined by an angle. By collecting these gamma rays and keeping track of the angles, or directions in which they were traveling, one can reconstruct an image that shows the distribution of radioactive material within the body.

For example, Single Photon Emission Computed Tomography (SPECT) imaging is performed by using a gamma camera to acquire multiple 2-D images (also called projections), from multiple angles. A computer is then used to apply a tomographic reconstruction algorithm to the multiple projections, yielding a 3-D dataset. This dataset can then be manipulated to show thin slices along any chosen axis of the body.

To acquire SPECT images, the gamma camera is rotated around the patient. Projections are acquired at defined points during the rotation, typically every 3-6 degrees. In most cases, a full 360 degree rotation is used to obtain an optimal reconstruction. The time taken to obtain each projection is also variable, but 15-20 seconds is typical. Therefore an angle of the gamma camera corresponds to a particular time or time interval.

The task of collecting these gamma rays is roughly analogous to placing cans outside during a rain shower and collecting drops. These “cans” are referred to as bins. To collect a meaningful number of “drops” (known as count) of gamma radiation in a bin can take a significant length of time. During this time, the patient must breathe. The act of breathing causes internal motion within the patient. This motion causes difficulties in image reconstruction. In particular, as a result of this motion, the reconstructed images may not show lesions, or they may include false lesions or other features.

It is possible to compensate for respiratory motion. Known methods of doing so involve collecting a signal that reflects the amplitude of the respiratory motion at a particular time (or angle of the gamma camera), and correlating that signal with the angles and counts detected by the gamma camera.

These known methods of motion compensation perform best when the patient's breathing is regular. However, in practice, the patient's breathing is anything but regular. At the beginning of the imaging procedure, a patient may be quite anxious. As the imaging procedure is carried out, the patient relaxes, and in some cases even falls asleep. Then, as time passes, the patient may awaken and become progressively more restless. These emotional states, which vary from patient to patient, and other factors may affect the regularity of the patient's breathing.

### SUMMARY

Motion estimation and compensation includes registration of a given image with a reference image. The present invention, is based, at least in part, on the discovery that if the reference image is somewhat degraded to make it more similar to a given image, then you can significantly reduce motion artifacts, e.g., due to breathing, to thereby improve the clarity and definition of nuclear images, e.g., SPECT images.

In one aspect, the invention features methods, e.g., computer-implemented methods, for compensating for respiratory motion of an individual during nuclear imaging of an object within the individual. The methods include defining a reference respiratory state for the individual; obtaining reference data representing the reference respiratory state; obtaining data representing a plurality of respiratory states that together correspond to at least a portion of a cycle of respiration of the individual; comparing data for each of the plurality of respiratory states to a subset of the reference data (which degrades the reference data) to obtain a motion estimate of each of the plurality of respiratory states; compensating for respiratory motion in each of the plurality of respiratory states based on the motion estimates to obtain motion-compensated respiratory state data; constructing an image using the motion-compensated respiratory state data; and providing an output representative of the image.

In these methods, the data representing the plurality of respiratory states can include projection data associated with imaging of an object within the individual at a plurality of discrete projection angles, and can further include respiratory signal data associated with respiration of the individual during the imaging.

The methods can further include associating the projection data at the discrete projection angles with a plurality of respiratory states of the individual to obtain amplitude binned respiratory state data for each of the plurality of respiratory states, wherein the plurality of respiratory states are defined based on amplitude levels of the respiratory signal data. In addition, in certain embodiments, the subset of the reference respiratory state data can include a first set of discrete projection angles selected from the discrete projection angles associated with the reference state data. This first set of discrete projection angles can be chosen based on common discrete projection angles associated with the reference data and data for a given respiratory state of the plurality of respiratory states.

In various embodiments, compensating for respiratory motion can include compensating on the basis of only the common discrete projection angles. In certain embodiments, defining the reference respiratory state can further include identifying a respiratory state having the greatest number of intact discrete projection angles. In some embodiments, reconstructing an image can include using a resealed block iterative system.

In another aspect, the invention features additional methods, e.g., computer-implemented methods, for compensating for respiratory motion of an individual during nuclear imaging of an object within the individual. These methods include receiving, in a computing device, projection data associated with imaging of the object at a plurality of discrete projection angles; receiving, in a computing device, respiratory signal data associated with respiration of the individual during the imaging; associating the projection data at the discrete projection angles with a plurality of respiratory states of the individual to obtain amplitude binned respiratory state data for each of the plurality of respiratory states, wherein the plurality of respiratory states are defined based on amplitude levels of the respiratory signal data; selecting a reference respiratory state from the amplitude binned data, the reference respiratory state including projection data at a first set of discrete projection angles; selecting at least one given respiratory state from the amplitude binned data, the at least one given respiratory state including projection data at a second set of discrete projection angles; determining a set of common projection angles from the first and second sets; and determining, by a computer, a relative position of the object in the given and reference respiratory states based on comparing three-dimensional reconstructions of the object from each of the given and reference respiratory states, wherein each reconstruction is based on projection data at the common projection angles.

In these methods, the imaging data associated with the object can include counts of photons emanating from the object and detected for each of the discrete projection angles in each respiratory state. The methods can further include dividing the photon count corresponding to a projection angle in a respiratory state by a scale factor. This scale factor can be calculated, for example, as a ratio of an actual acquisition time for the projection angle to a predetermined length of time. For example, the predetermined length of time can be representative of an ideal acquisition time.

In certain embodiments, the methods can further include using the scale factor as a threshold to determine whether the data acquired from the object at the projection angle should be used for a three-dimensional reconstruction of the object. I various embodiments, selecting the reference respiratory state can further include determining a number of intact projection angles for each of the respiratory states, and defining the reference respiratory state can further include selecting the reference respiratory state at least in part on the basis of the number of intact projection angles associated with the reference respiratory state.

In some embodiments, the reference respiratory state can be defined by identifying the respiratory state having the greatest number of intact projection angles. In some embodiments, determining the relative position of the object in the given and reference respiratory states can further include registering the three-dimensional reconstruction of the object in the given state to the three-dimensional reconstruction of the object in the reference state, and comparing three-dimensional reconstructions of the object in each of the given and reference respiratory states can include comparing corresponding two dimensional slices from the three-dimensional reconstructions.

In certain embodiments, reconstructing an image can include using a rescaled block iterative system or using a maximum-likelihood expectation maximization (MLEM) system.

The new methods can be carried out in conjunction with various nuclear imaging systems such as SPECT systems. Thus, in another aspect, the invention features systems that include a nuclear imaging system configured to image an object within an individual; a respiratory sensor configured to generate a respiratory signal based on respiration of the individual; and a processor configured to carry out the steps of any the methods described herein. In these systems, the nuclear imaging systems can be or include SPECT imaging systems, and the respiratory sensor can be or include an air filled girdle or pneumatic bellows.

In another aspect, the invention also features computer-readable media storing a computer program for compensating for respiratory motion of an individual in nuclear imaging of an object. These computer programs include instructions for causing a computer system or processor to carry out any one or more of the steps of the methods described herein. In some embodiments, the computer system is caused to define a reference respiratory state for the individual; obtain reference data representing the reference respiratory state; obtain data representing a plurality of respiratory states that together correspond to at least a portion of a cycle of respiration of the individual; compare data for each of the plurality of respiratory states to a subset of the reference data to obtain a motion estimate of each of the plurality of respiratory states; compensate for respiratory motion in each of the plurality of respiratory states based on the motion estimates to obtain motion-compensated respiratory state data; construct an image using the motion-compensated respiratory state data; and provide an output representative of the image.

In another aspect, the invention also features computer-readable media storing a computer program for compensating for respiratory motion of an individual in nuclear imaging of an object. These computer programs include instructions for causing a computer system to receive projection data associated with imaging of the object at a plurality of discrete projection angles; receive respiratory signal data associated with respiration of the individual during the imaging; associate the projection data at the discrete projection angles with a plurality of respiratory states of the individual to obtain amplitude binned respiratory state data for each of the plurality of respiratory states, wherein the plurality of respiratory states are defined based on amplitude levels of the respiratory signal data; select a reference respiratory state from the amplitude binned data, the reference respiratory state including projection data at a first set of discrete projection angles; select at least one given respiratory state from the amplitude binned data, the at least one given respiratory state including projection data at a second set of discrete projection angles; determine a set of common projection angles from the first and second sets; and determine a relative position of the object in the given and reference respiratory states based on comparing three-dimensional reconstructions of the object from each of the given and reference respiratory states, wherein each reconstruction is based on projection data at the common projection angles.

The invention provides numerous benefits and advantages (some of which may be achieved only in some of its various aspects and implementations) including the following. In general, the invention improves the quality of registration and 3-D reconstruction for cardiac SPECT imaging especially by reducing errors due to respiratory motion. For example, the invention can significantly improve the ability to detect lesions and other defects. In addition, the invention avoids or reduces artifacts due to false cooling and limited angle reconstructions. As a result, the corrected images obtained using the new systems and methods can be used by physicians for diagnosis and treatment with increased reliability.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic of a nuclear imaging system.

FIG. 2A is a graphical representation of the amplitude of a respiratory signal over time.

FIG. 2B is a representation of projection angles associated with bins in an exemplary scheme of amplitude binning.

FIG. 3 is a flow chart of exemplary operations in a motion estimator.

FIG. 4 is a schematic of a subsystem for processing data for a nuclear imaging system.

FIG. 5A is a block diagram representing a motion estimator.

FIG. 5B is a block diagram of a motion compensated (e.g., a motion compensated maximum-likelihood expectation maximization or MCMLEM) reconstruction.

FIGS. 6A and 6B are a pair of graphs showing registration errors that arise using a conventional motion estimation method.

FIGS. 6C and 6D are SPECT coronal slice images to be registered when using the conventional motion estimation method.

FIGS. 7A and 7B are a pair of graphs showing the significant reduction in registration errors that arise using the motion estimator of FIG. 5A.

FIGS. 7C and 7D are SPECT coronal slice images to be registered when using the motion estimator of FIG. 5A.

FIGS. 8A-8F are graphs showing motion estimation results for different configurations of a NCAT phantom. These motion estimates are taken as a function of number of angles retained in the reference state.

FIGS. 9a-9j are representations of short axis slices from a NCAT configuration with a perfusion defect.

FIGS. 10a-10j are representations of short axis slices from a NCAT configuration with altered angulation and no perfusion defect.

FIG. 11A-11I are a series of graphs that show acquired time for 34 different projection angles for each of nine respiratory states for a given first patient.

FIGS. 12A and 12B are representations of short axis slices corresponding to the patient noted in the description of FIGS. 13A-13I.

FIGS. 13A-13I are a series of graphs that show acquired time for 34 different projection angles for each of nine respiratory states for a second patient.

FIGS. 14A and 14B are representations of short axis slices corresponding to the second patient noted in the description of FIGS. 13A-13I.

### DETAILED DESCRIPTION

The inventions described herein can be implemented in many ways. Some useful implementations are described below. The descriptions of implementations of the inventions are not descriptions of the inventions, which are not limited to the detailed implementations described in this section, but are described in broader terms in the claims.

System Overview

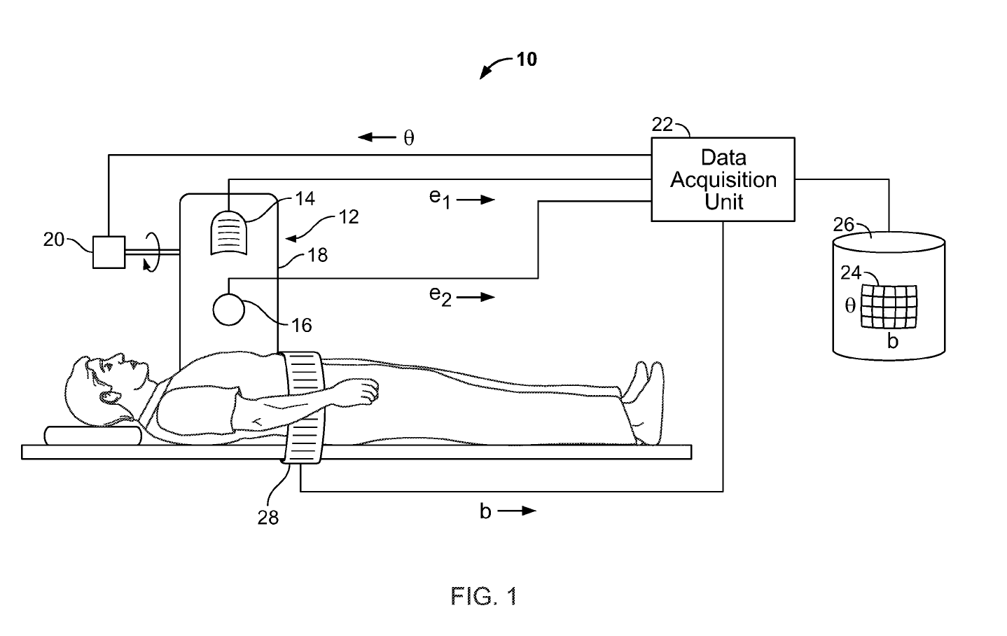

FIG. 1 shows a system 10 for collecting data to be corrected by methods and systems described herein. The system 10 features a nuclear imaging subsystem 12, such as a SPECT system, having a pair of Anger cameras or gamma cameras 14, 16 mounted on a gantry 18 configured for rotation by an actuator 20.

A gamma camera 14, 16, consists of one or more flat crystal planes (or detectors) optically coupled to an array of photomultiplier tubes. This assembly is known as a “head” which is mounted on the gantry 18. The gantry 18 is connected to a computer system that both controls the operation of the camera as well as acquisition and storage of acquired images. The system accumulates events, or counts, of gamma photons that are absorbed by the crystal in the camera. Usually a large flat crystal of sodium iodide with thallium doping in a light-sealed housing is used. Scintillations are produced in response to gamma radiation incident on the crystal. A gamma photon emanating from a patient (who has been injected with a radioactive pharmaceutical) knocks an electron loose from atom in the crystal (iodine in this example). A faint flash of light is produced when the dislocated electron again finds a minimal energy state. The detectors in the gamma cameras 14, 16 detect the flash of light. In some cases, photomultiplier tubes (PMTs) behind the crystal detect the fluorescent flashes (events) and a computer sums the counts. The computer reconstructs and displays a two dimensional image of the relative spatial count density on a monitor. This reconstructed image reflects the distribution and relative concentration of radioactive tracer elements present in the organs and tissues imaged.

A data acquisition unit 22 provides a control signal (θ) for causing rotation of the gantry 18, and hence the cameras 14, 16, about a patient. As the gantry 18 rotates, the Anger cameras 14, 16 provide event data e1, e2 to the data acquisition unit 22 for storage in a data table 24 on a mass storage device 26.

A respiratory sensor 28 on the patient's abdomen provides a respiratory signal b to the data acquisition unit 22 during the procedure. A typical respiratory sensor 28 is an air filled girdle, or pneumatic bellows, whose volume decreases during a patient's inhalation, thus changing the air pressure within the girdle, and thereby providing a basis for monitoring respiration. This signal can then be used to bin the list-mode data provided by the cameras 14, 16.

The amplitude of the respiratory signal b provided by the respiratory sensor 28 defines a set of bins, with each bin corresponding to a different portion of the patient's respiratory cycle. The data acquisition unit 22 knows the times at which events e1, e2 occur as well as the times at which the patient was in a particular respiratory state. As a result, it is possible for the data acquisition unit 22 to associate each event with a particular portion of the respiratory cycle. This information can be used as a basis for correcting for motion caused by patient respiration and achieving registration across different points of the respiration cycle.

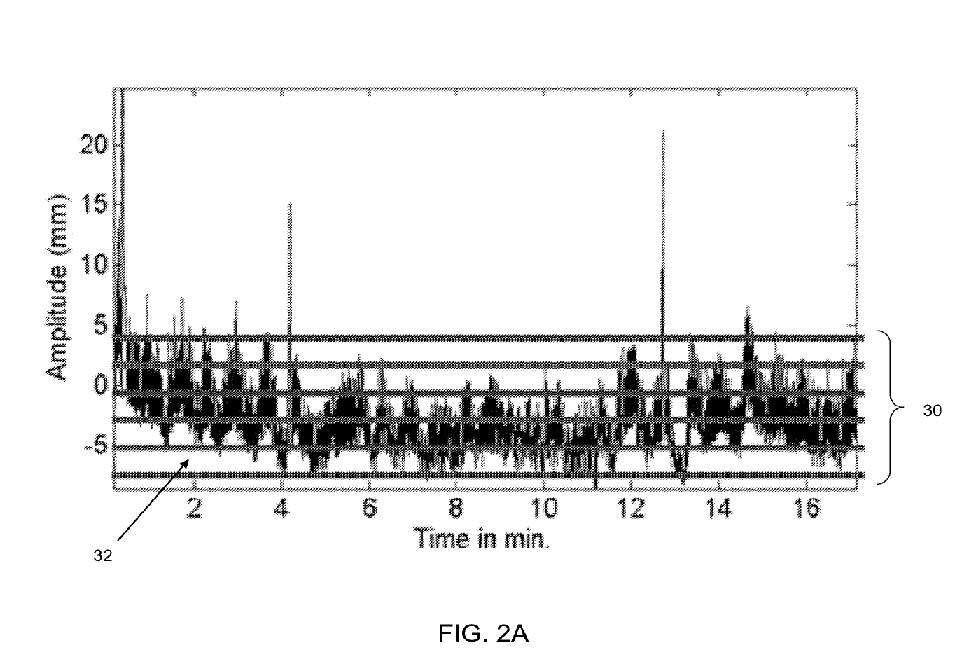

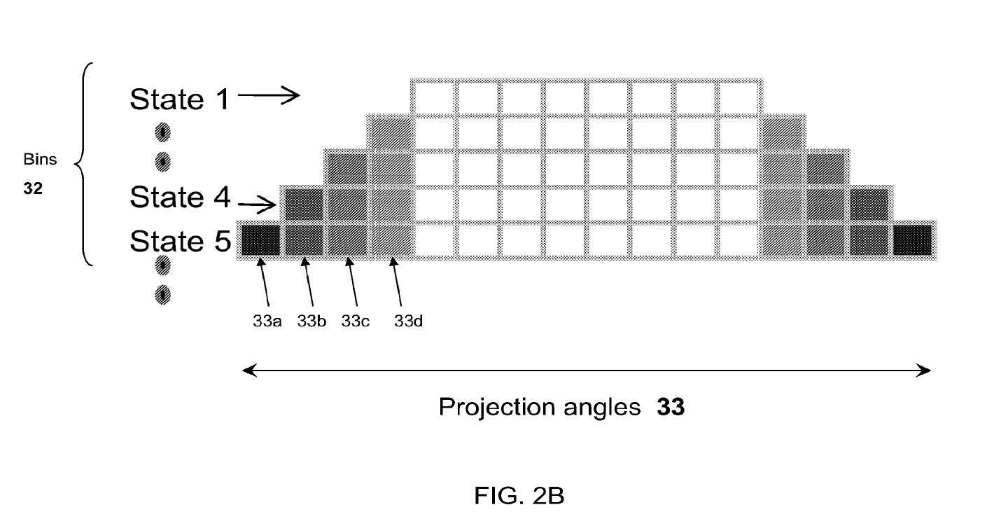

FIG. 2A shows a plot of the respiratory signal amplitude as a function of time. Amplitude bins 30 are shown as parallel horizontal lines. It is apparent from FIG. 2 that because the patient's breathing is not entirely regular, the signal spends varying amounts of time in each bin 30. For example, for the first four minutes, the signal spends essentially no time in the bin 32 extending between −5.0 mm and −7.5 mm.

Since the position of various internal organs changes during the respiratory cycle, each amplitude bin 30 corresponds to a particular position of the internal organs, referred to herein as the “respiratory state.” If very few counts are available in a particular bin 32, it will naturally be more difficult to reconstruct a good image of the internal organs at that point in the respiratory cycle. As a result, there would be very few counts available to reconstruct an image corresponding to that portion of the respiratory cycle. Thus, when estimating the motion by comparing between the reconstructions at different bins, it becomes necessary to compare dissimilar reconstructions. This difficulty arises because there is no guarantee that all the bins will have the same or similar counts for all the angles, or even any count for some angles.

This result is illustrated in FIG. 2B by way of an example, in which only 5 respiratory states have been shown for illustrative purposes. Each row in FIG. 2B represents a bin, which in turn corresponds to a particular respiratory state. Each column in FIG. 2B represents a particular projection or acquisition angle 33. Each individual square represents an individual projection image for a given state and a given angle. Progressively higher numbers of counts are represented by progressively darker shade of gray. In other words, a light shade of gray depicts a small number of counts while a darker shade represents a relatively higher count. If there are no counts or a low number of counts at certain bins for a projection angle, there will be compensatory counts in the other bins for the corresponding angle. This is due to the fact that the object continues to emit photons and the total acquisition time is the same for each angle. In other words, the total number of counts in each column 33 will be substantially the same. In this example, the counts for a given projection angle are evenly distributed among the bins for the corresponding angle. Therefore in effect the further out projections will have more and more counts, as depicted by the progressively darker shades of gray.

The problem of unequal number of counts in different bins 32 is exacerbated by the fact that each count in a bin is associated with a particular projection angle. Each projection angle is preset to acquire events for the same length of time. Thus, even as the patient's respiration causes the heart to move across the different amplitude bins 32, there may be very few counts available in a particular bin. For example, referring again to FIG. 2B, the number of counts in the bin corresponding to state 1 is significantly lower than the number of counts in the bin corresponding to state 5.

In general, a count is never lost. Instead, it is simply misplaced. As a result, counts that should have been placed in one bin are instead placed in a different bin. This result exacerbates the error since not only is one bin shortchanged, but the counts are further unevenly distributed in other bin(s).

Known methods of motion compensation ignore the fact that some bins 32 may have very few counts corresponding to certain angles. In known methods, one simply determines whether a bin has enough counts to be worthwhile processing. If so, the bin is processed, even though there may be a paucity of counts associated with certain angles. If the bin has insufficient counts, it is not processed at all. This amounts to throwing away information that might otherwise be exploited. For example, the bin corresponding to state 1 in the example of FIG. 2B can be discarded in prior methods due to a very small number of counts detected in the bin.

In some implementations of the new inventions, motion estimation is carried out by identifying a reference state and identifying the motion between a given state and the reference state by taking into account only a subset of the projection angles that are available in a reference bin. In effect, the extent of motion between a reference state and a given state is determined by degrading the data associated with the reference state to match the data associated with the given state.

General Methodology

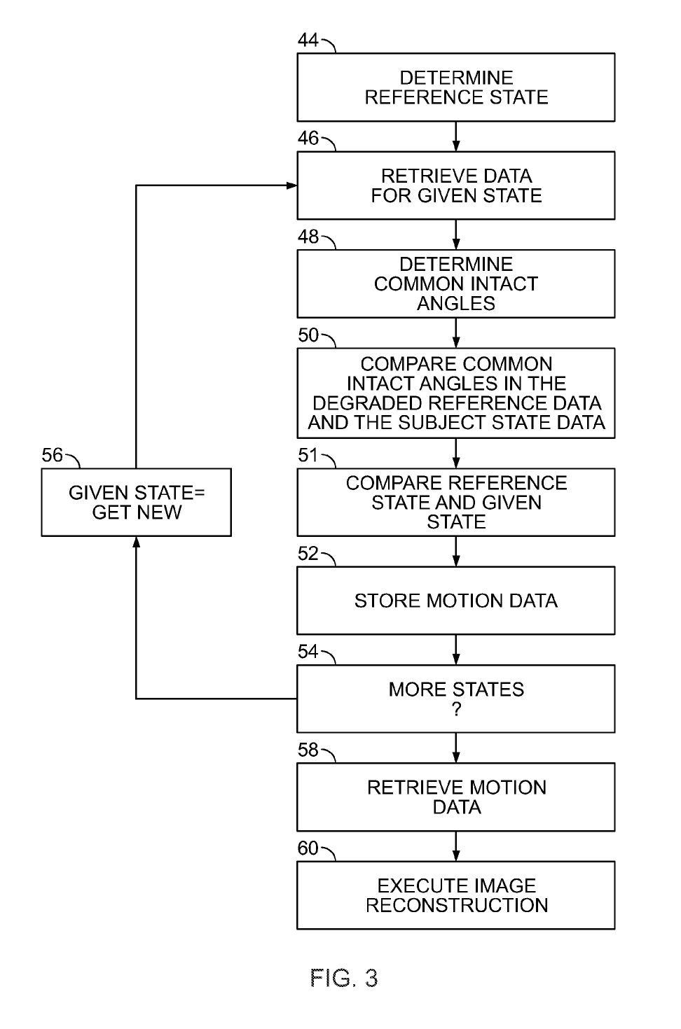

FIG. 3 is a flow chart of an exemplary method carried out by in a motion compensation system. The illustrated method begins by determining which of the various respiratory states is to be designated as the reference state (step 44). Once the reference state is determined, data corresponding to a given state is retrieved (step 46). Once the reference state and data given state are both available, it becomes possible to determine the common intact angles (step 48), to compare the reference state and the given state only at those common angles (steps 50 and 51), and to store the results of the comparison (step 52). In general, the number of intact angles in the reference state is more than that of the given state. Determining the common intact angles between the given and reference states can therefore include degrading the reference state by excluding some intact angles from the reference state. Such a counterintuitive degrading of the reference state improves the quality of registration with the given state by preprocessing the reference state to resemble the given state more closely. If there are additional respiratory states (step 54), the foregoing procedure is repeated for the next state (step 56). Otherwise, the motion data is retrieved (step 58) and used to execute image reconstruction (step 60).

The reference state (step 44) can be determined in a number of ways. In some implementations, the reference state can be chosen based on binning list-mode data acquired for different respiratory states using an external motion tracking device such as the respiratory sensor 28 described with respect to FIG. 1. Binning can be based either on the relative position within the respiratory cycle (phase binning) or the amplitude of the signal from the external motion tracking device 28 (amplitude binning). The following discussion is based on amplitude binning unless otherwise specified. However, phase binning can also be used in some implementations without deviating from the scope of this application. Typically, the state or bin that has the largest number of intact angles is chosen as the reference bin. For example, referring again to FIG. 2, the bin corresponding to respiratory state 5 can be chosen as the reference bin.

The data for the given state is retrieved (step 46) and preprocessed for comparison with the reference state. Because each respiratory state corresponds to a particular bin in the binned list-mode data, the terms “state” (or “respiratory state”) and “bin” are used interchangeably throughout this application. In some implementations, three dimensional (3D) reconstructions are carried out for both the given and reference states before comparing the states. In some implementations, comparing involves registration of the 3D data corresponding to the given state to the 3D data corresponding to the reference state. In some implementations, a two dimensional (2D) slice or segment of the reconstructed 3D data is used in the registration. As mentioned above, for a given angle in the binned data there may be unequal counts across the different respiratory states. This result may be due to irregular breathing of the patient or other movements associated with the patient. In some implementations, unequal numbers of counts in different states for a given angle can be preprocessed as follows.

Normalization of Counts Across Different States for Each Angle

In some implementations, the total number of counts corresponding to a projection angle for a given state (i.e., an individual projection image) is divided by a scale factor based on the acquisition time for that particular angle. The acquisition time data are available from the time stamps in the list-mode data. Such time stamps are used in amplitude binning to synchronize the SPECT data collection with the data from the external motion tracking device 28. In some implementations, the scale-factors are calculated as:

$$scalefactor = \frac{Δ {t}_{actual}}{Δ {t}_{even}} \qquad ( 1 )$$

where Δt_{actual} is the actual acquisition time for the individual projection image and Δtₑᵥₑₙ is the acquisition time per projection image assuming the ideal case of even or equal sampling across all states for the given angle. Δtₑᵥₑₙ is therefore given by:

$$Δ {t}_{even} = \frac{total acquistiontime}{( no . of angles ) \times ( no . of bins per angle )} \qquad ( 2 )$$

In some implementations, the number of counts in each projection image is divided by its corresponding scale-factor. This process normalizes the counts across the projection images such that the normalized counts approximate the number that would have been acquired if each projection image had been acquired for the same amount of time. In some implementations, further processing can be carried out based on the calculated scale-factor. For example, for a given state, if the calculated scale-factor for a given angle is less than a pre-determined threshold (say 0.5 or 0.3, 0.4, 0.6, or 0.7), the counts of the corresponding projection image can be set to zero. In other words, a thresholding based on the calculated scale factor is carried out to reject projection images with insignificant number of counts. The factor, e.g., 0.5, is used purely for illustrative purposes and should not be considered limiting. Rather, the scale-factor based threshold can be determined, sometimes empirically, based on the quality of motion estimation and/or reconstruction desired. In some implementations, such thresholding can be used to restrict noise amplification and/or increase a signal to noise ration (SNR). The thresholding can also be used to exclude certain projection images not contributing to the slices from which the estimation of respiratory motion is performed. Thresholding can also result in excluding projection images with both very low counts as well as no counts. In general, such projection images lead to limited-angle reconstruction artifacts that interfere with the estimation of motion.

Motion Estimation with Limited Angles

In some implementations, the common intact angles between the given and reference states are determined (step 48) to enhance meaningful comparison between the states. For example, if the given state has projection images corresponding to very few angles as compared to the reference state, the difference between the corresponding images are dominated by distortions due to the different view angles. Such distortions can degrade the motion estimate, in some cases, severely. In some implementations, such distortions can be overcome by using projection images in the reference state corresponding to the available angles in the given state. For example, consider registration of state 4 with a reference state 5 of FIG. 2B. Since the angle 33a is missing from the state 4, the registration process will omit the corresponding projection image from sate 5 and instead use only the common angles between the states. In this way the limited-angle effects are substantially the same between the two states. The reference data with the omitted angles may be referred to as the degraded reference data.

In some cases, when very few or even no common angles exist between two respiratory states under consideration, the first state can be registered to an intermediate respiratory state whose motion compared to the reference state has been estimated. In such cases, the motion of the first state relative to the reference state is obtained as a combination of the motion estimate between the reference state and the intermediate state and the motion estimate between the first state and the intermediate state. For example, referring again to FIG. 2B, since very few common angles exist between state 2 and the reference state 5, the state 2 can first be registered with respect to, for example, state 4. The state 4, in this example, will be the intermediate state. If the motion estimate of state 2 with respect to state 4 is 0.2 cm and the motion estimate of state 4 with respect to state 5 is 0.5 cm, the motion estimate of state 2 with respect to state 5 will be given by 0.5 cm+0.2 cm=0.7 cm.

The common intact angles, the degraded reference data and the data for the given state are compared (step 50) to estimate the motion between the states. In some implementations, the comparison is carried out by way of registering 3D reconstruction data corresponding to the two states. For cardiac SPECT imaging, this includes reconstructing 3D images or data sets of the heart corresponding to the respiratory states being registered. Any 3D reconstruction algorithm, such as the maximum-likelihood expectation maximization (MLEM) algorithm, can be used for the reconstruction. The reconstruction algorithms use the projection images corresponding to the common intact angles to reconstruct 2D images that make up the 3D images corresponding to the given and reference states. In general, the 3D data is an assembly of ordered 2D images. The 2D images that make up the 3D representation may be referred to as “slices.”

The given and the reference states are compared (step 51) to estimate the motion between the two states. In some implementations, the motion is estimated based on registering a slice from the 3D representation of the given state to the corresponding slice from the 3D representation of the reference state. The process may then be repeated for each corresponding pair of slices from the 3D representations of the two states. In some implementations, the 3D data sets can be registered with respect to each other.

Any image registration algorithms can be used for the registration process. Typically, a registration algorithm matches a plurality of corresponding points between two data sets using a similarity metric. This can be viewed as a process of transforming the different sets of data into one coordinate system to be able to compare or integrate the data obtained from different measurements. Registration includes transforming a first data set in multiple ways to determine an orientation with respect to a second data set. The transformations could include, without limitation, translation, rotation, shear and scale. In general, the different number of transformations governs the number of degrees of freedom associated with the registration algorithm. The registration point or orientation is determined when a similarity metric is optimized. In some implementations, an intensity based registration algorithm is used to align the given state and the reference state. The similarity metric “sum of squared differences” (SSD) can be used for the registration. In such cases, the two data sets are registered at the point when the SSD between the pixel (or voxel) intensities is minimized. The SSD is given by:

$$S S D = {\sum }_{n} {[ {I}_{1} ( x , y , z ) - {I}_{2} ( T ( x , y , z ) ) ]}^{2}$$

where I₁(x, y, z) are the intensities in the reconstructed reference dataset and I₂(x, y, z) are the intensities in the reconstructed dataset for the state to be registered to the reference. Other metrics of similarity such as sum of absolute differences (SAD), correlation coefficient, root mean squared differences (RMS) and mutual information (MI) can also be used. In some implementations, an optimization metric can be used to minimize the similarity metric such as the SSD. For example, gradient descent can be used as the optimization metric. In such cases analytical gradients can be calculated to increase efficiency.

In some implementations, the transformation T associated with the registration algorithm is a 12 degree-of-freedom (DOF) affine transformation with three scales, three shears, three rotation and three translation components. Other transformations with varying degrees of freedom can also be chosen without deviating from the scope of this application. The choice of the transformation can depend on the nature of the images or datasets that are sought to be registered. For example, in cardiac SPECT imaging, the respiratory motion of the heart during quiet supine breathing is mostly in the superior/inferior (SI) direction followed by the anterior/posterior (AP) direction. In such cases, the medial/lateral translation and rotations about any of these axes may be negligible.

The motion estimate or motion data for a particular state is stored (step 52) at locations such as the mass storage device 26. In some implementations, the system checks to see if there are additional states (step 54) for which motion estimation is to be performed. The system can also have prior knowledge about the number of states and can proceed to process data related to additional states without performing the checking step. If there are additional unprocessed states, the system retrieves a new given state (step 56) and repeats the above steps for the new given state.

After motion estimation data for all steps are calculated and stores, the system generates a motion compensated 3D dataset from the data related to the respiratory states using the motion estimates calculated in the steps described above. In some implementations, generation of the motion compensated 3D dataset includes retrieving the stored motion estimate data (step 58) and executing an image reconstruction algorithm (step 60) based on the motion estimates as well as the acquired projection data for the different states.

In some implementations, all the original or un-normalized projection images are used along with the calculated motion estimates to generate the final motion compensated reconstruction of the object (for example the heart, in case of cardiac SPECT). In other words, in some implementations, counts from projection images are not discarded or scaled for the final reconstruction. In some implementations, one or more of attenuation correction, resolution compensation by modeling of the system response and iterative reconstruction can be used for the final reconstruction in addition to the motion compensation. Any reconstruction algorithm, such as a motion-compensated MLEM (MCMLEM) algorithm can be used for the reconstruction. Additional details on the MCMLEM algorithm are described in the following publication, the entire content of which is incorporated herein by reference: Joyoni Dey and Michael King, “Theoretical and Numerical Study of MLEM and OSEM Reconstruction Algorithms for Motion Correction in Emission Tomography,” IEEE Trans. On Nuclear Science, Vol. 56, No. 5, pages 2739-2749, October 2009.

In some implementations, an iterative reconstruction algorithm is used. The equation for an example iterative reconstruction algorithm is given by:

$${f}^{n + 1} ( i ) = {f}^{n} ( i ) \frac{{\sum }_{b} {Q}_{b}^{0} [ {\sum }_{j ∈ {S}_{b}} \frac{{c}_{ij} {Y}_{j}^{b}}{{\sum }_{i} {c}_{ij} {f}^{n} ( {M}_{0}^{b} ( i ) )} ]}{{\sum }_{b} {Q}_{b}^{0} [ {\sum }_{j ∈ {S}_{b}} {c}_{ij} ]} \qquad ( 3 )$$

where M is a transformation of spatial index and Q is a motion operator operating on the intensities. In other words, M₀^{b}(i) transforms the index i to go from reference pose 0 to pose at State b. The operator Q is defined as, (Q₀^{b}f)(i)=f(M₀^{b} (i)) and S_{b} is the set of angles with non-zero counts for the respiratory State b. The counts in each bin (unevenly distributed or not) are considered, moved to the reference position, and added together.

Hardware Subsystem

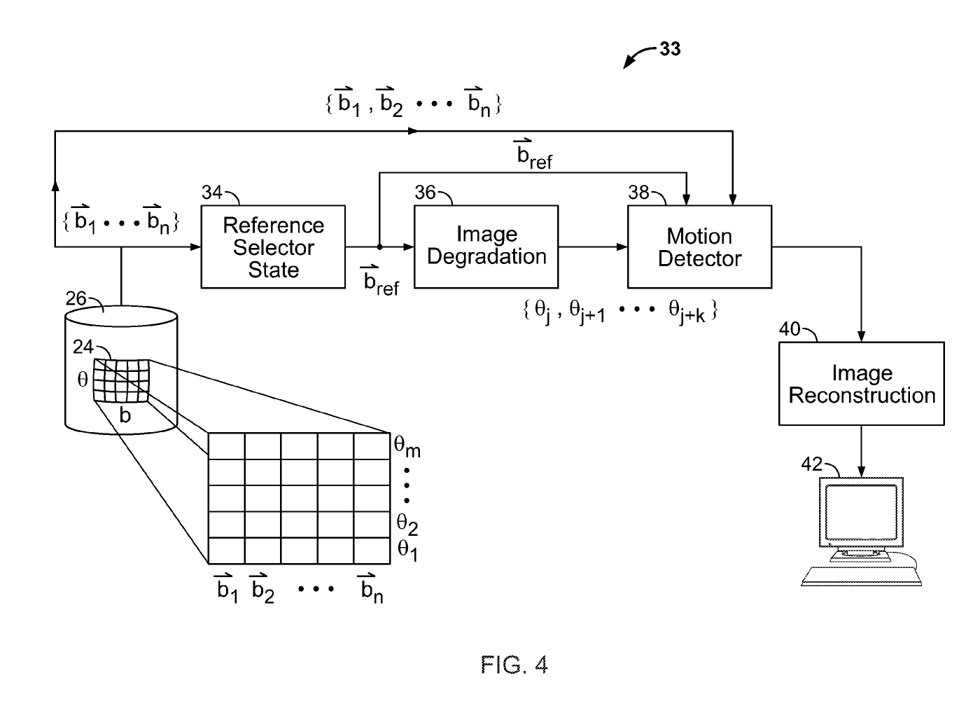

FIG. 4 shows a subsystem 33 for implementing the operations described above with respect to FIG. 3. In a broad overview, the subsystem 33 includes a computer-readable medium having instructions that, when executed by a computer, define certain modules that retrieve data 24 from a mass storage device 26, perform various manipulations of that data 24, and then output a tangible result, e.g., an image of an organ, such as the heart, on a display 42. Alternatively, the image can be printed in various ways, or transmitted electronically to another device or display located at a distance from the nuclear imaging subsystem 12. The tangible image is one that can be relied upon by a physician in connection with diagnosis and treatment of the patient.

In general, the features described herein can be implemented in digital electronic circuitry, or in computer hardware, firmware, or in combinations of them. The features can be implemented in a computer program product tangibly embodied in an information carrier, e.g., in a machine-readable storage device, for execution by a programmable processor; and features can be performed by a programmable processor executing a program of instructions to perform functions of the described implementations by operating on input data and generating output. The described features can be implemented in one or more computer programs that are executable on a programmable system including at least one programmable processor coupled to receive data and instructions from, and to transmit data and instructions to, a data storage system, at least one input device, and at least one output device. A computer program includes a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. Computers include a processor for executing instructions and one or more memories for storing instructions and data. Generally, a computer will also include, or be operatively coupled to communicate with, one or more mass storage devices 26 for storing data files; such devices include magnetic disks, such as internal hard disks and removable disks; magneto-optical disks; and optical disks. Mass storage devices 26 suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

The mass storage device 26 has stored thereon a table 24 of bins 32, each of which corresponds to a stage in the respiratory cycle, and a listing of counts associated with each of a plurality of angles. The bins are collectively represented as vectors {b₁, b₂, . . . , bₙ}. A reference-bin-selection module 34 identifies the bin (represented as the vector b_{ref}) that has the most intact angles. As used herein, an angle is “intact” if the number of counts associated with that angle is in excess of a predetermined threshold.

An image-degradation module 36 then inspects the intact angles of a given bin and the intact angles {θⱼ, θⱼ₊₁, . . . , θⱼ₋ₖ} of the reference bin. On the basis of that inspection, the image-degradation module 36 identifies the angles to be used during comparison between the reference bin and given bin for motion estimation purposes. Specifically, the angles used in such comparison are restricted to only those angles that are intact angles in both the reference respiratory state (bin) and the given respiratory state (bin). An angle that is intact in both the reference respiratory state and the given respiratory state is a “common intact-angle.”

The common intact-angles, the reference-bin data, and the data for the given-bin are then provided to a motion detector 38, which then reconstructs the data and calculates a suitable registration between the given respiratory state and the reference respiratory state. In some implementations, the reconstruction method is Resealed Block Iterative (RBI) Algorithm, which can be used to account for the differences in the counts between bins even for a subset of retained angles within a bin. In some implementations, the reconstruction algorithm is a statistical reconstruction algorithm such as the maximum-likelihood expectation maximization (MLEM) algorithm.

Once all the bins have been processed, the image is reconstructed using the estimated motion obtained in the previous stage and all the angles available for all the bins, using any conventional motion-compensated iterative reconstruction technique 40 such as the motion compensated MLEM algorithm. The resulting image is then output for display on a display device 42.

To provide for interaction with a user, the features can be implemented on a computer where the display device 42 is a CRT (cathode ray tube) or LCD (liquid crystal display) monitor for displaying information to the user. Alternatively, the tangible image can be printed in a variety of ways, or transmitted to another device, such as a hand-held computer or personal digital assistant (PDA). The computer can have a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

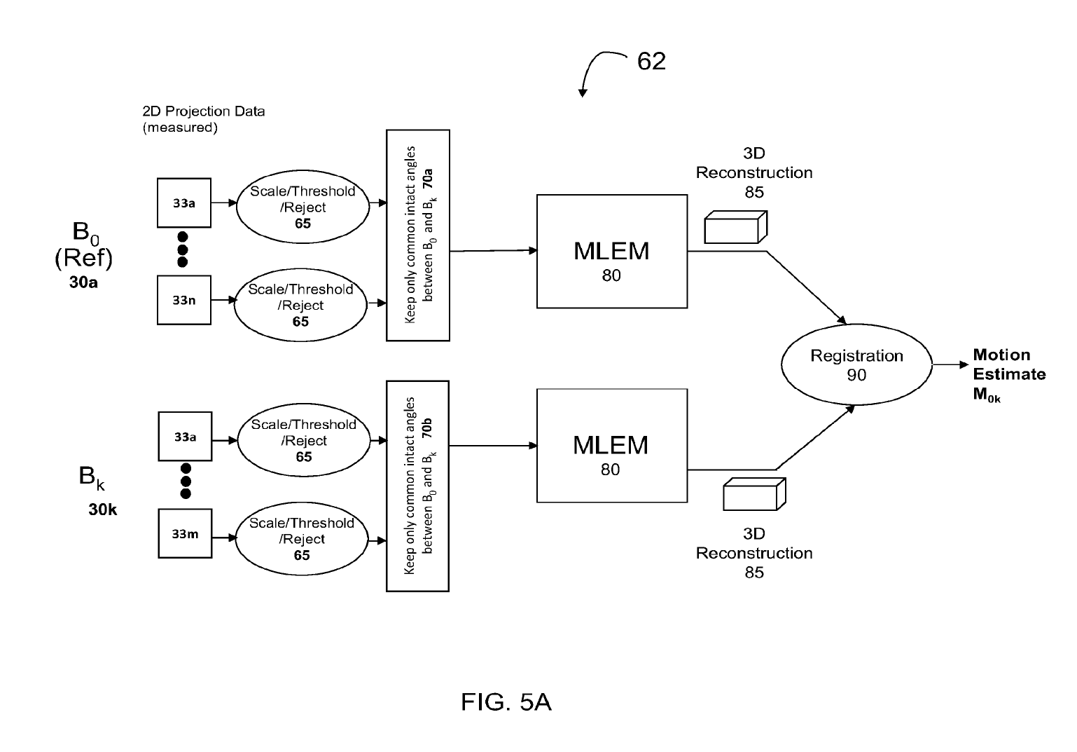

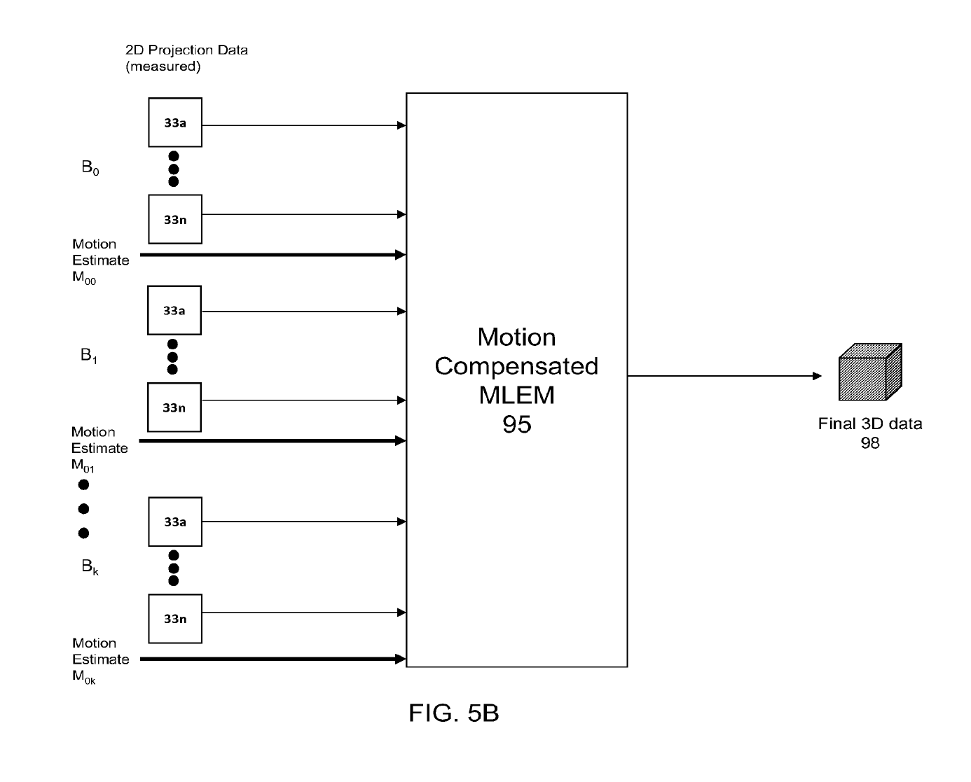

The steps described above with respect to FIG. 3 are summarized in the block diagrams shown in FIGS. 5A and 5B. Referring to FIG. 5A, a block diagram of a system 62 for estimating motion between a reference state B₀ 30a and a given state Bₖ 30k is shown. The reference state 30a includes individual projection images 33a-33n (33 in general), each corresponding to a particular angle of acquisition. The given state 30k also includes individual projection images 33a-33m (33 in general), each corresponding to a particular angle of acquisition. In general, the number of projection images 33 in the reference state 30a will be greater than that corresponding to the given image 30k. The counts in each of the projection images 33 are scaled and/or thresholded and/or rejected in the block 65. The scaling, thresholding, and rejecting is done as described with respect to FIG. 3. In block 70a, projection images corresponding to certain angles from the reference state 30a are rejected. In some implementations, angles that are not present in the given state 30k are rejected. Similarly, in block 70b, angles from the given state 30k which are not present in the reference state 30a are rejected or excluded from further processing. In other words, only the common angles between the given and reference states are retained for both states.

2D projection images corresponding to the remaining angles are used for MLEM reconstruction 80. A 3D reconstruction 85 is obtained for each state and is subject to registration 90. A motion estimate is obtained as a result of registering the 3D reconstructions with each other. In this example, since the motion estimate is obtained between the reference state B0 30a and a given state Bk 30k, it is denoted as M_{0K}.

FIG. 5B shows a block diagram for a MCMLEM reconstruction system 95. In some implementations, the system 95 accepts the 2D projection images 33 from each state B₀-Bₖ as well as the corresponding motion estimates M₀₀-M₀ₖ. The system 95 outputs a 3D dataset 98 which is a motion compensated rendition of the object being imaged. For example, in cardiac SPECT imaging, the final 3D data 98 will be a motion compensated image of the heart.

### EXAMPLES

The following examples are merely descriptive and not intended to limit the various inventions described herein, which are embodied in the pending claims.

Simulation of Cardiac SPECT Imaging with NCAT Phantom: Setup

Studies were conducted using simulation of Tc-99m sestamibi cardiac-perfusion imaging. The source and attenuation distributions were created using the NURBS-based cardiac-torso (NCAT) phantom 16. The NCAT phantom was specifically modified to allow different extents of respiratory motion of the heart and other structures, such as liver, around it. The relative concentration of activity in the liver, gall-bladder, kidney, spleen was half of that of the heart. The background was 1/10 that of the heart. Distributions for a total of 36 respiratory states were created equally subdividing a 2 cm extent of motion of the heart in the SI direction and 6 mm in the AP direction. These ranges were selected to match the ranges observed during actual imaging and also to allow for motion due to upward creep of the heart. The standard angulation of the heart was used for the NCAT (NCAT parameters of −90 deg, −20 deg, and −50 deg about x, y and z-axes). This configuration is referred to as Configuration I.

Additionally, a variation of the standard configuration in which a large (clinically relevant) perfusion defect was added in the lateral wall of the basal region (Configuration II), was also investigated. In this configuration, the angular extent of the defect along the left-ventricular wall on the short axis is 60 deg and the length of the defect is 60 mm in the long-axis direction. The perfusion defect was introduced to investigate the impact of respiratory motion on visibility of defects. Also investigated was a third configuration (Configuration III) of the NCAT phantom in which the orientation of the heart is nearly perpendicular to the superior-inferior (SI) axis of the body (NCAT parameters of −90 degree, 0 degree, and 0 degree about x, y and z-axes, respectively). This positioning of the heart provides maximum sensitivity to blurring of the anterior and inferior walls of the heart in the presence of axial motion and thus provides an excellent test-case for motion compensation.

SIMIND Monte Carlo software, made available by Lund University, Sweden, was used to simulate a typical clinical 2-headed SPECT acquisition and generate noiseless projections for 36 respiratory states. Each of the two heads of the SPECT system was simulated to acquire 30 evenly spaced projections over 90 degree for a total of 60 projections over 180 degree. Projection size was 128×128 pixels with a pixel dimension of 4.67 mm. A low-energy-high-resolution (LEHR) parallel collimator was simulated.

Acquisition was simulated for a 15% photopeak window centered on the photon emission energy of Tc-99m. The projection images contained both the primary and scattered photons that were recorded within this window. A total of 433.56 million photons/projection were simulated, thereby yielding very low-noise projections.

A setup substantially same as that described above was used for the experiments in examples 1-3 described below.

### Example 1

### Motion Estimation with Low Noise NCAT Projections

The impact of limited-angle reconstruction on motion estimation was investigated in the low-noise projection sets created by SIMIND using all three configurations of the NCAT phantom. This investigation was done to study biases introduced by estimating motion between slices reconstructed using an increasingly different number of projection angles. When testing the effects on estimation due to missing angles, groups of adjacent angles from different parts of the orbit were rejected for each head. In some cases, large chunks of missing angles can cause bigger problems than angles missing at random. Referring again to FIG. 2A, time varying trends in respiration can lead to chunks of missing angles. Since images are acquired by two heads (Head I and Head II) simultaneously, angles missing from one head in general implies the corresponding angles missing from the other. The simulation was configured to account for this.

The 18th (near middle) respiratory state was selected as the reference state and the 36^{th} state, which had maximum variations from the reference, was selected as the test respiratory state. All angles were used to reconstruct the slices of the reference state and a lesser number of angles were used to reconstruct the slices of the test state. In this example, the number of angles retained (NAR) from Head I for reconstruction of test slices were reduced from 30 in steps of 2 by removing 1 from each end till only 2 angles remained. Matching changes in the NAR were made for head 2. For each NAR (varying from 30 to 2 in steps of 2), the slices of the test respiratory state were reconstructed and the motions in the superior/inferior (SI) and anterior/posterior (AP) directions estimated in each case. For the reference respiratory state, all 30 angles were retained for both heads. The estimated motions were then compared to the true motions used for creating the NCAT distributions. Since there were 36 respiratory states and state 18 was chosen as the reference, the true motions between states 36 and 18 were −10.3 mm in SI and 3.1 mm in AP. Reconstructions were done using standard MLEM with correction for distance-dependent system spatial-resolution collimator, but without attenuation or scatter correction. This process was then repeated using the same NARs for the reference and test respiratory states.

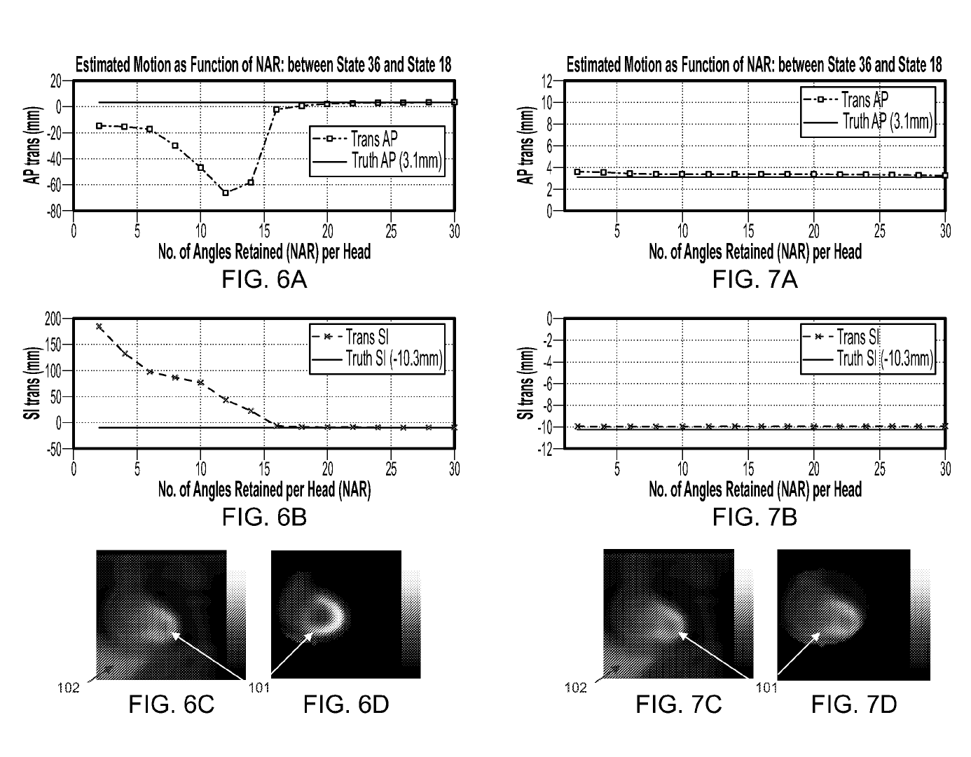

Referring now to FIGS. 6A and 6B, registration results for motion estimation of the reference state 18 with respect to state 36 are shown. All angles in the reference state were used for reconstruction and the angles for state 36 were varied from 30 (all angles/head) to just two angles remaining in the middle of the orbit. The AP (FIG. 6A) and SI (FIG. 6B) motions were estimated and compared against the actual motions (truth). The motion estimates are seen to deviate from the truth (both for AP and SI) for less than 16 NAR per head. For example, as seen in FIG. 6B, for NAR=2, the registration error rises to nearly 200 mm in the SI direction. This shows that using a relatively higher number of angles for the reference is not desirable when only a limited number of angles is available for the given state.

FIGS. 6C and 6D illustrate the problem. FIG. 6C shows a coronal slice for the reconstruction of respiratory state 36 with 2 angles retained per head. FIG. 6D shows the corresponding slice from the reconstruction of reference respiratory state 18 using all available angles. In the reference state (FIG. 6D), the heart is segmented whereas it is not so for the given state 36 (FIG. 6C). In other words, only the heart 101 is visible in FIG. 6D and not any other organ such as the liver 102 as seen in FIG. 6C. The dissimilarity between the corresponding images leads to large registration errors. The dissimilarity can be more pronounced in trans-axial slices.

FIGS. 7A and 7B show the motion estimation results when the reference state is reconstructed using only the angles present in the given state. In this low-noise example the registration achieved sub-millimeter accuracy. FIGS. 7C and 7D show that matching the corresponding angles in reconstructing the reference and given states make the images to be registered more similar to each other as compared to the case depicted in FIGS. 6C and 6D. For example, the image for the reference state (FIG. 7D) is seen to be visually similar to the image for the given state (FIG. 7C) except for differences due to motion. The above results indicate that the quality of registration is improved, both qualitatively and quantitatively, using the methods and systems described herein.

### Example 2

### Motion Estimation in Presence of Poisson Noise in NCAT Projections

A set of experiments using the first configuration of the NCAT was performed with added noise. Since dividing the counts from a SPECT cardiac acquisition into 36 respiratory states would be too noisy, the 36 low-noise projection sets were combined in groups of 4 to create 9 motion states. Noise was added to the projections for each state. Reducing the 36 states into 9 resulted in each of the 9 states being a combination of counts from the structures of the NCAT in 4 slightly different spatial distributions. This approximately simulated real cases where continuous variation in motion is sampled. The expected motion was re-calculated for the 9 states (taking into account quantization). For the extreme states, the expected motions were now +/−9.1 mm for the SI direction and +/−2.7 mm for the AP direction. The NAR was varied from 30 to 2 in steps of 2 for each head. The above steps were carried out for the extreme respiratory states 1 and 9, state 5 being the reference state. Registration was performed between states 1 and 5 for each of the 15 NAR at the start, middle, and end of acquisition. The process was then repeated for states 9 and 5. For these experiments only reconstructions with corresponding angles retained in the reference and given states were compared. The counts were evenly divided between the 9 respiratory motion states at a given angle so that states 1, 5, and 9 each contained 1/9 the counts acquired at the given angle.

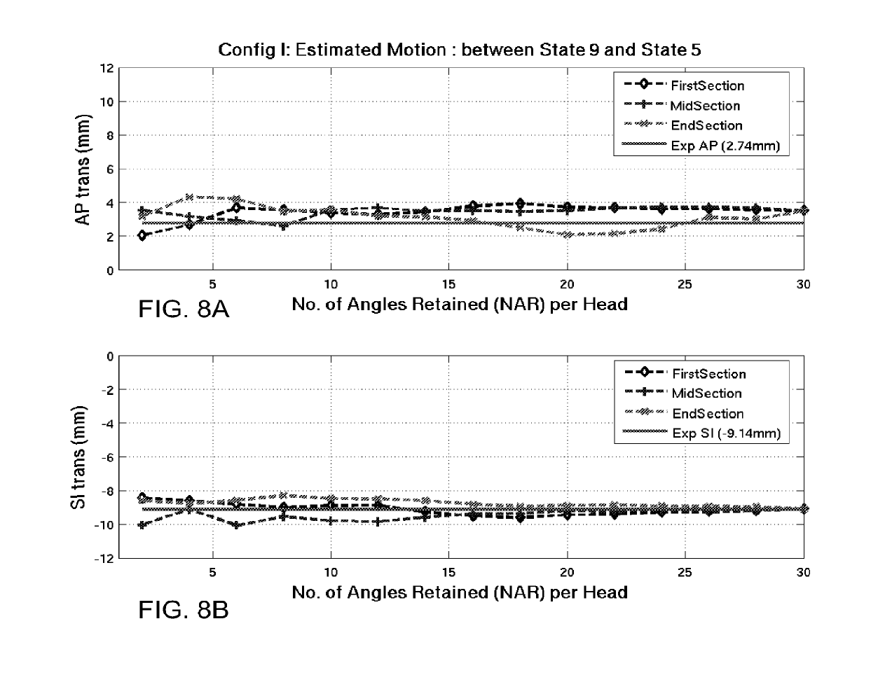

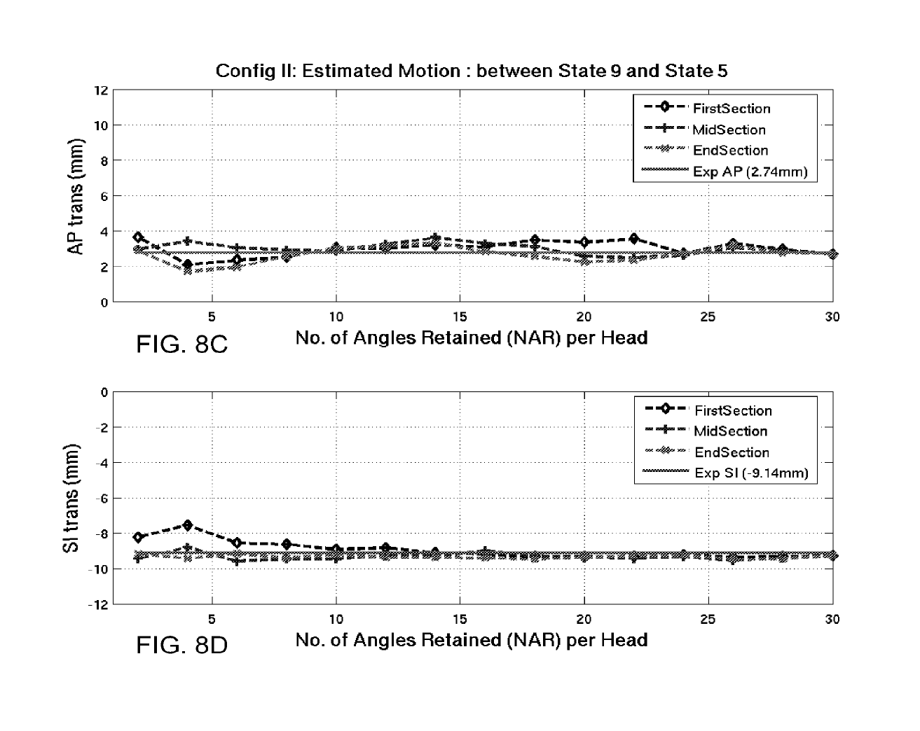

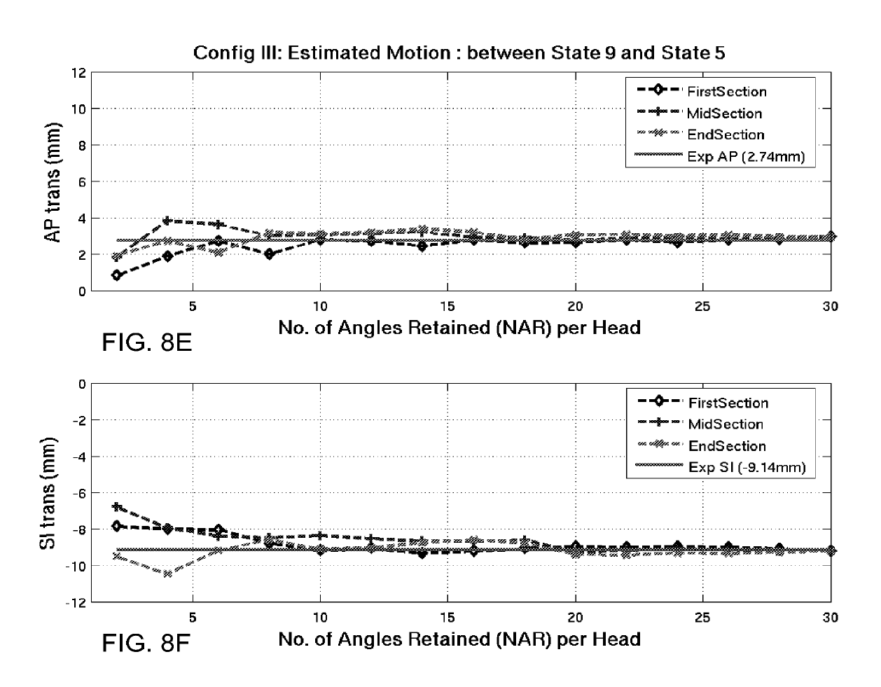

FIGS. 8A-8F show results of motion estimation between the reference state 5 and state 9 for the three configurations of the NCAT phantom (without/with defect and with a different angulation of the heart) when clinically relevant Poisson noise is added in the simulated slices. In each figure either the estimated AP (FIGS. 8A, 8C and 8E) or SI (FIGS. 8B, 8D and 8F) motion is plotted as a function of the NAR when the angles are retained in the first, mid, or end sections of the acquisition for each head.

Quantitative results of motion estimation for both extreme states 1 and 9 relative to the reference state are summarized in Tables I-III for the three configurations of the NCAT phantom. The average RMS error (=\sqrt{Yₑᵣᵣ²+Zₑᵣᵣ²}) (where Y denotes the AP translation and Z denotes SI translation) is shown, and the average is computed over the number of angles retained (NAR). The AP and SI errors are calculated as the difference from the expected values of +/−9.1 mm for SI and +/−2.7 mm for AP.

TABLE 1
Default Angulation of heart (Config I)

| | FirstSection | MidSection | EndSection |
|---|---|---|---|
| ConfigI | Mean RMS | Mean RMS | Mean RMS |
| (default) | Error (mm) | Error (mm) | Error (mm) |
| States 1&5 | 1.03 | 1.14 | 1.20 |
| States 9&5 | 0.85 | 0.85 | 0.76 |

TABLE II
With Defects (Config II)

| | FirstSection | MidSection | EndSection |
|---|---|---|---|
| Config II (with | Mean RMS | Mean RMS | Mean RMS |
| defects) | Error (mm) | Error (mm) | Error (mm) |
| States 1&5 | 0.81 | 0.74 | 0.54 |
| States 9&5 | 0.60 | 0.44 | 0.44 |

TABLE III
With Different Angulation of the Heart (Config III)

| Config III | | | |
|---|---|---|---|
| (different | FirstSection | MidSection | EndSection |
| angulation of | Mean RMS | Mean RMS | Mean RMS |
| the heart) | Error (mm) | Error (mm) | Error (mm) |
| States 1&5 | 1.45 | 1.30 | 1.57 |
| States 9&5 | 0.49 | 0.67 | 0.55 |

The above tables indicate that in these examples, the maximum average RMS error over all three configurations was only 1.57 mm, thereby showing the effectiveness of the methods and systems described herein.

### Example 3

### Motion Correction in NCAT Simulations

Three examples of limited-angle data (denoted LAD1 to LAD3) were created for the second and third configuration of the NCAT phantom. For LAD1 the NAR per head across the 9 respiratory states were 14, 18, 22, 26, 30, 26, 22, 18, and 14, respectively. In other words, the NAR decreased in steps of 4 from the extreme states to the reference state. LAD2 represented a case where the NAR decreased in steps of 6 from the extreme states to the reference state. Therefore, the NAR per head for the 9 states were 6, 12, 18, 24, 30, 24, 18, 12, and 6, respectively. In case of LAD3 the step size was 6 resulting in the NAR of 2, 8, 16, 24, 30, 24, 16, 8, and 2, respectively. In all three cases, the angles retained were distributed in the mid-section of the projection angles acquired by each head. A fourth case, denoted by LAD0 was created for comparison purposes and included all 30 projection angles present for the 9 states for each head.

In addition to the missing angles, a change in the number of counts was also simulated within the binned projections for a given respiratory state. For simplicity the number of counts acquired at a given angle was equally divided among all states. Poisson noise was added and the net count over all projection images was simulated to be 7.5 million. Motion estimation and reconstruction was carried out as described with respect to FIG. 3. For comparison purposes, the motion correction was redone with the expected motions for each bin. Resolution compensation and attenuation correction were also used in the reconstruction. For attenuation correction, the average attenuation map over all bins was used.

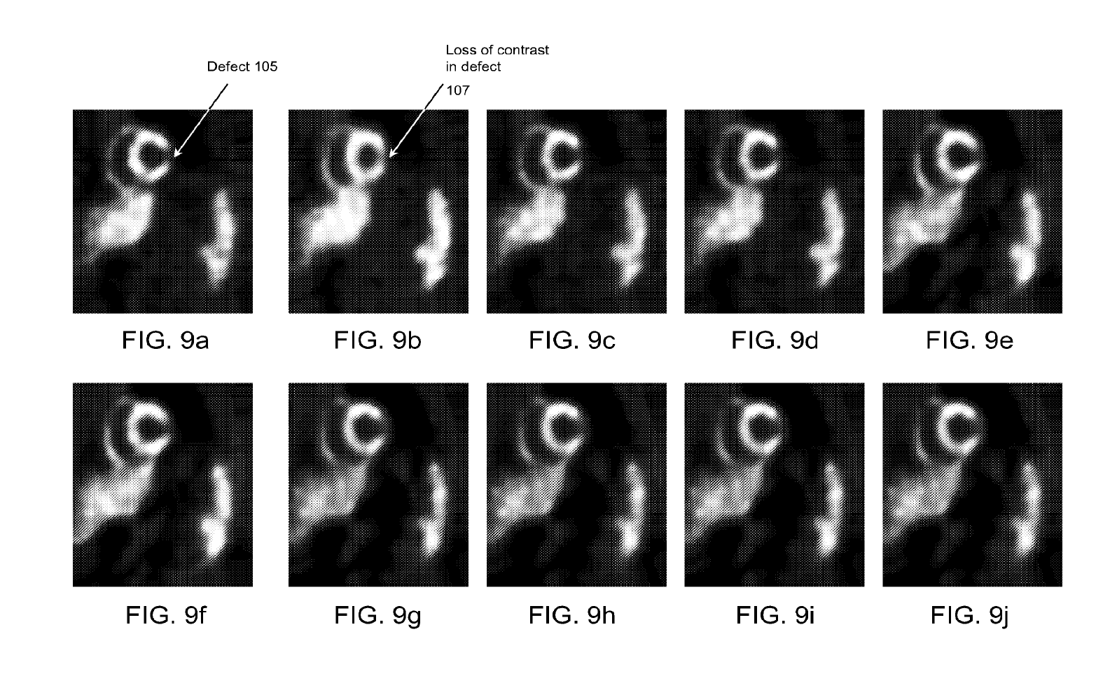

Each of the FIGS. 9a-9j shows a short-axis slice, for the second NCAT configuration (with defect 105). FIG. 9a shows the reference state reconstructed with total count restored to 7.5 million. It thus served as the standard for successful respiratory motion correction. FIG. 9b shows the reconstruction of the projections obtained by summing all the respiratory states for each angle. FIG. 9b shows the impact of uncorrected respiratory motion and exhibits a loss of contrast 107 for the lateral defect. FIG. 9b also exhibits a decrease in size of the blood pool region within the left-ventricle (LV), the thickening of the walls of the LV, and the blending of the LV with the liver due to uncorrected respiratory motion. FIGS. 9c and 9d show the cases where the motion was compensated during reconstruction using the expected values of motion and estimated values of motion, respectively, for each state in LAD0 (i.e. all 30 angles intact for each head). In both FIGS. 9c and 9d the alterations caused by motion are largely corrected. FIGS. 9e and 9f show the LAD1 simulations reconstructed with expected and estimated motions, respectively. Significant improvements are seen in these cases as compared to FIG. 9b. FIGS. 9g-9h and 9i-9j show similar results for LAD2 and LAD3 simulations, respectively.

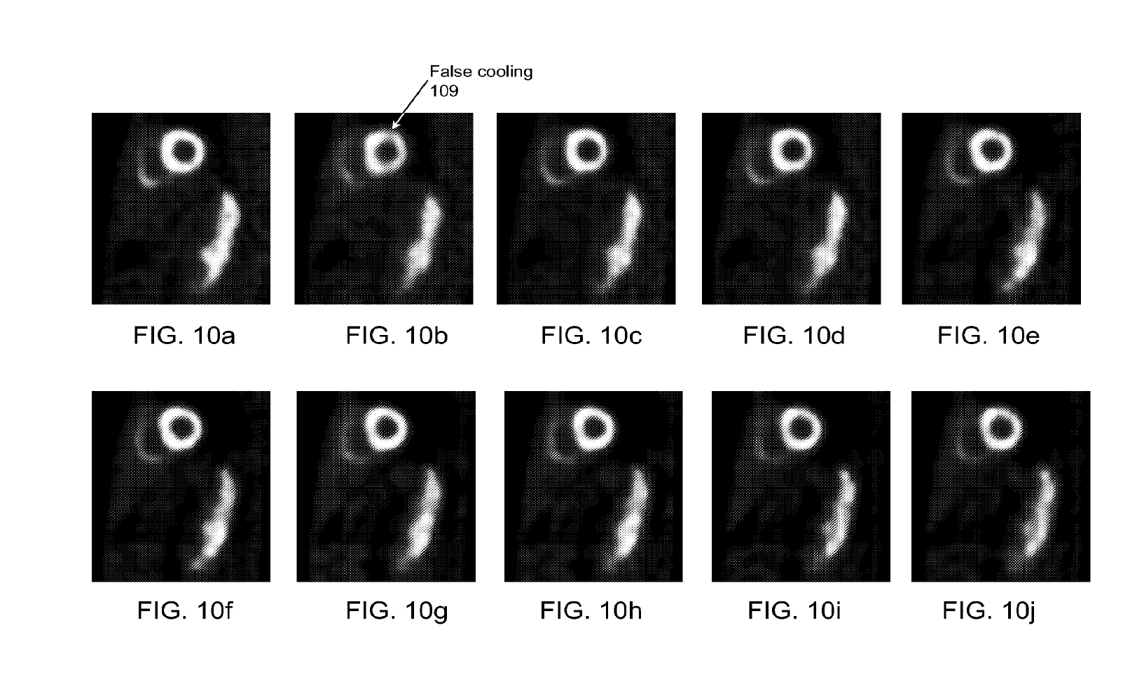

FIGS. 10a-10i show the results for the third NCAT configuration with no added defect, but with an alteration in the angulation of the heart as compared to the standard NCAT angulation parameters. Specifically FIG. 10b shows the case of reconstruction without motion compensation. FIG. 10b exhibits artifacts due to false cooling 109 in the superior and anterior walls. These artifacts could potentially be mistaken as defects. The motion correction algorithms demonstrated by FIGS. 10c-10j (shown for LAD0-LAD3 with expected and estimated motion) are seen to reduce these artifacts.

### Example 4

### Motion Correction of Clinical Acquisitions

Under institutional review board (IRB) approval and with informed consent of patients, clinical studies were conducted on several patients. These patients were subjected to ^{99m}Tc sestamibi cardiac-perfusion imaging. A bellows was placed around the abdomen of each patient at the umbilical level. The placement and adjustment of the bellows required approximately one minute to accomplish, was well tolerated by the patients, and was the sole modification to the standard imaging protocol as far as the patient was concerned.

Software provided by Philips Medical Systems of Cleveland, Ohio was used to enable list-mode acquisition on a triple-head IRIX® SPECT system. Counts, time stamps, and gantry angles (for tomographic acquisition) were recorded in the list-mode files. In some cases, external analog and digital events can also be interspersed within the list. The data was sequentially stored in the list-mode file in an order of occurrence. For each count, the information stored included: camera head detecting the photon, count location in a 2048×2048 matrix, and photon energy. A time stamp was written every 10 ms. This duration defined the temporal resolution for all events. Only the step-and-shoot gantry rotation mode was available with list-mode acquisition. Thus data was not acquired during rotation.

A non-metallic pneumatic bellows from Lafayette Instruments Co. of Lafayette, Ind. was used as the respiratory sensor 28 and wrapped around the patients' abdomens. The bellows was connected to a pressure transducer manufactured by Lafayette Instruments Co. of Lafayette, Ind. The transducer was attached to a laptop through a DAQCard-6062E acquisition card manufactured by National Instruments Co. of Austin, Tex. Variations in pressure due to the stretching and compression of the bellows with respiration resulted in a variation in the recorded voltage from the pressure transducer. This was the respiratory signal used for amplitude binning the list-mode counts as a function of respiration.

The LabVIEW programming environment, from National Instruments Co., Austin, Tex., was used to create a graphical user interface (GUI) for recording the respiratory signal at 10 Hz, i.e., one measurement from the bellows every 100 ms. In addition, the LabVIEW GUI was used to trigger the start of the SPECT acquisition and send a synchronization signal. The synchronization signal consisted of a repeating pattern of +3 volt and −3 volt levels, with a period of 8 seconds, to an analog input on the SPECT system for recording in the list-mode file. By knowing the time at which the synchronization signal was sent relative to the recording of the respiratory signal, a temporal matching was established between the respiratory signal recorded by the laptop and the emission events recorded in the list-mode file on the SPECT system.

The cardiac-stress list-mode acquisitions were performed on IRIX gamma-camera using low-energy high-resolution collimators approximately 60 minutes after an injection of 925-1110 MBq (25-30 mCi) of 99 mTc-sestamibi. The acquisition duration was 19.8 seconds per projection. Sixty-eight projections were acquired with two heads over 204 degrees in total, with the two heads 102 degrees apart. The first camera head started at 123 degrees (approximately right-anterior oblique in terms of patients) relative to the posterior direction. Step and shoot body-contouring gantry motion was employed and the acquisition time was approximately 15 minutes. Events were recorded within a symmetric energy window centered at 140 keV and having a width of 15%. Sequential transmission imaging using the scanning 133 Ba point sources of the Beacon system (Philips Medical Systems, Cleveland, Ohio) was employed for the estimation of the attenuation maps.

Software was developed to bin the 2048×2048 list-mode data into a set of 128×128 frames of 100 ms at each projection angle with a pixel size of 4.67 mm. For the acquisition duration of 19.8 seconds per projection, this resulted in 198 projections of 100 ms for each projection angle. The 100 ms duration was chosen to match the frequency of acquisition of the respiratory signal. The 100 ms projections were sorted into 9 respiratory states according to the amplitude of the respiratory signal at the time the projection was acquired. The respiratory signal data was thresholded to remove infrequent extreme values. Values beyond the thresholding limits were replaced by the respective limiting values. The range of the remaining values was divided into 9 uniform size intervals or states between the maximum (corresponding to the maximum inspiratory volume) and minimum (corresponding to the maximum expiratory volume). The choice of 9 states was empirical. Using fewer than 9 would have increased the coarseness of binning and more than 9 would have decreased the signal to noise ratio. For each projection angle, the 100 ms projections, whose respiratory states fell in the same interval, were summed to produce 9 projections for that angle, one for each of the 9 states of respiratory motion. The variability in acquisition time for a given state with a projection angle was analyzed to assess the extent of the potential problem with angular representation in the projections sets. The counts in the projection bins were then normalized and the motion between the states estimated as previously described. After motion estimation, the un-normalized projections for each state were used along with the motion estimates in iterative reconstruction to obtain motion compensated reconstructions. Short-axis slices, with and without respiratory motion correction, were compared visually as an index of the extent of change resulting from motion correction.

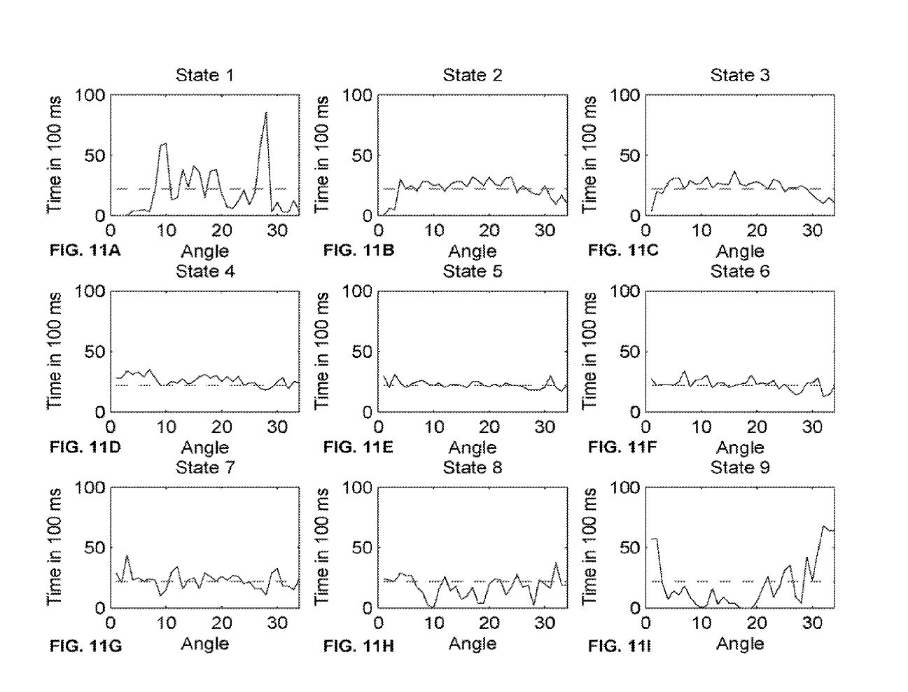

FIGS. 11A-11I show the acquired times for each binned state for a first cardiac SPECT patient. The times are shown in units of 100 ms. The acquisition times in the different motion states are seen to vary significantly as a function of the projection angle (time point during acquisition). The horizontal dashed line in each case shows the acquisition time if the breathing was perfectly regular throughout the acquisition period. This served as the threshold in these experiments. As described with respect to FIG. 3, if the acquired times for a given angle and state fell below a certain fraction (one half in this case) of this value, that angle was not included in the reconstruction for motion estimation. Accordingly, certain states exhibited more dependence on the NAR than the others. For example, states 1 (FIG. 11A), 8 (FIG. 11H) and 9 (FIG. 11I) were found to be affected the most by NAR. States 2 (FIG. 11B) and 3 (FIG. 11C) were moderately affected while states 4-7 (FIGS. 11D-11G) were the least affected by NAR.

A 6-DOF affine transformation based registration was used to estimate the motion between a given state and the reference state. State 5 was chosen to be the reference state. Only SI and AP translations were found to be non-negligible.

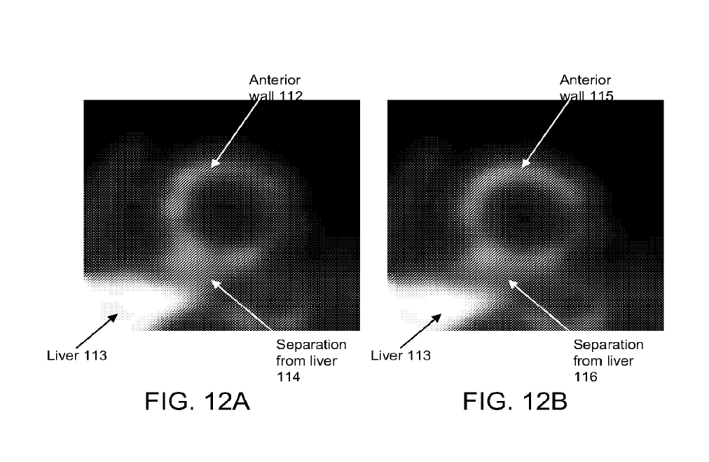

The motion estimation results were used for the motion compensated reconstruction. The degree of improvement of the resultant images depended on a variety of factors such as extent of motion, counts in the extreme states, wall thickness, and orientation of the heart with respect to the direction of motion. FIG. 12A shows a short-axis slice for the first patient without motion correction. FIG. 12B shows the same shorts axis slice with motion correction. The alteration in appearance was consistent with that observed with the NCAT simulations but was less marked due to the small magnitude of motion. FIG. 12B shows a brightening of the anterior wall 115 as compared to the anterior wall 112 of FIG. 12A, a better definition of the lateral defect and an increased separation 116 from the liver 114.

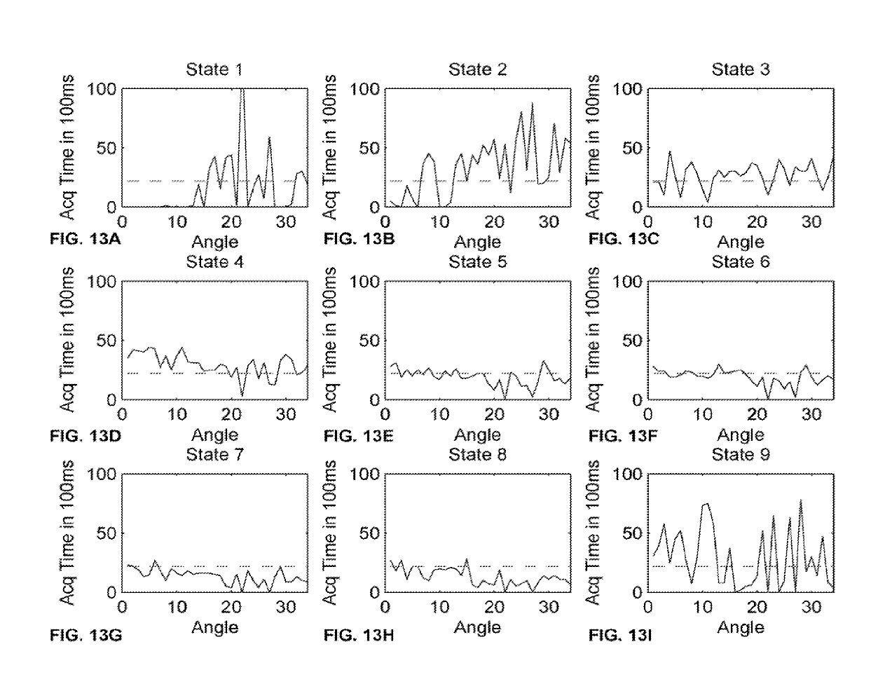

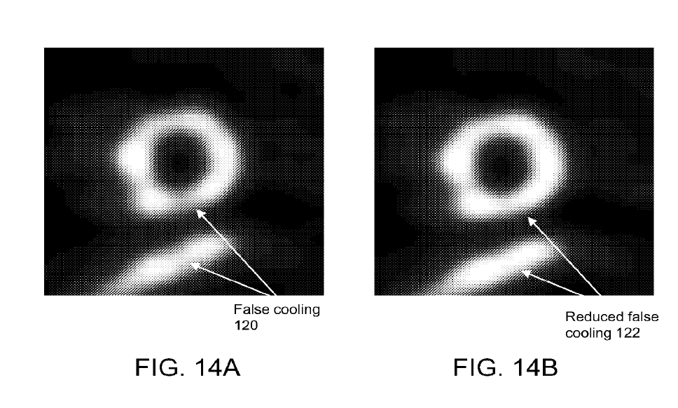

FIGS. 13A-13I shows the acquired times for each binned state for a second cardiac SPECT patient. In this case states 1 (FIG. 13A), 8 (FIG. 13H) and 9 (FIG. 13I) were seen to be most affected by NAR and states 2 (FIG. 13B) and 7 (FIG. 13G) were moderately affected. FIGS. 14A and 14B show corresponding short axis slices with and without motion correction, respectively. Referring to FIG. 14A, a false cooling 120 in the inferior wall and a hint of a defect in the anterior wall are observed. Referring to FIG. 14B, these artifacts are seen to be reduced with motion compensation. For example, a reduced false cooling 122 is observed in FIG. 14B.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A computer implemented method for compensating for respiratory motion of an individual during nuclear imaging of an object within the individual, the method comprising:
defining a reference respiratory state for the individual;
obtaining reference data representing the reference respiratory state;
obtaining data representing a plurality of respiratory states that together correspond to at least a portion of a cycle of respiration of the individual;
comparing data for each of the plurality of respiratory states to a subset of the reference data to obtain a motion estimate of each of the plurality of respiratory states;
compensating for respiratory motion in each of the plurality of respiratory states based on the motion estimates to obtain motion-compensated respiratory state data;
constructing an image using the motion-compensated respiratory state data; and
providing an output representative of the image.

2. The method of claim 1, wherein the data representing the plurality of respiratory states comprises projection data associated with imaging of an object within the individual at a plurality of discrete projection angles.

3. The method of claim 2, wherein the data representing the plurality of respiratory states further comprises respiratory signal data associated with respiration of the individual during the imaging.

4. The method of claim 3, further comprising associating the projection data at the discrete projection angles with a plurality of respiratory states of the individual to obtain amplitude binned respiratory state data for each of the plurality of respiratory states, wherein the plurality of respiratory states are defined based on amplitude levels of the respiratory signal data.

5. The method of claim 2, wherein the subset of the reference respiratory state data comprises a first set of discrete projection angles selected from the discrete projection angles associated with the reference state data.

6. The method of claim 5, wherein the first set of discrete projection angles are chosen based on common discrete projection angles associated with the reference data and data for a given respiratory state of the plurality of respiratory states.

7. The method of claim 6, wherein compensating for respiratory motion comprises compensating on the basis of only the common discrete projection angles.

8. The method of claim 1, wherein defining the reference respiratory state further comprises identifying a respiratory state having the greatest number of intact discrete projection angles.

9. The method of claim 1, wherein reconstructing an image comprises using a rescaled block iterative system.

10. A computer-implemented method for compensating for respiratory motion of an individual during nuclear imaging of an object within the individual, the method comprising:
receiving, in a computing device, projection data associated with imaging of the object at a plurality of discrete projection angles;
receiving, in a computing device, respiratory signal data associated with respiration of the individual during the imaging;
associating the projection data at the discrete projection angles with a plurality of respiratory states of the individual to obtain amplitude binned respiratory state data for each of the plurality of respiratory states, wherein the plurality of respiratory states are defined based on amplitude levels of the respiratory signal data;
selecting a reference respiratory state from the amplitude binned data, the reference respiratory state comprising projection data at a first set of discrete projection angles;
selecting at least one given respiratory state from the amplitude binned data, the at least one given respiratory state comprising projection data at a second set of discrete projection angles;
determining a set of common projection angles from the first and second sets; and
determining, by a computer, a relative position of the object in the given and reference respiratory states based on comparing three-dimensional reconstructions of the object from each of the given and reference respiratory states, wherein each reconstruction is based on projection data at the common projection angles.

11. The method of claim 10, wherein the imaging data associated with the object includes counts of photons emanating from the object and detected for each of the discrete projection angles in each respiratory state.

12. The method of claim 11, further comprising dividing the photon count corresponding to a projection angle in a respiratory state by a scale factor.

13. The method of claim 12, further comprising calculating the scale factor as a ratio of an actual acquisition time for the projection angle to a predetermined length of time.

14. The method of claim 13, wherein the predetermined length of time is representative of an ideal acquisition time.

15. The method of claim 12, further comprising using the scale factor as a threshold to determine whether the data acquired from the object at the projection angle should be used for a three-dimensional reconstruction of the object.

16. The method of claim 10, wherein selecting the reference respiratory state further comprises determining a number of intact projection angles for each of the respiratory states.

17. The method of claim 16, wherein defining the reference respiratory state further comprises selecting the reference respiratory state at least in part on the basis of the number of intact projection angles associated with the reference respiratory state.

18. The method of claim 16, wherein defining a reference respiratory state further comprises identifying the respiratory state having the greatest number of intact projection angles.

19. The method of claim 10, wherein determining the relative position of the object in the given and reference respiratory states further comprises registering the three-dimensional reconstruction of the object in the given state to the three-dimensional reconstruction of the object in the reference state.

20. The method of claim 10, wherein comparing three-dimensional reconstructions of the object in each of the given and reference respiratory states comprises comparing corresponding two dimensional slices from the three-dimensional reconstructions.

21. The method of claim 10, wherein reconstructing an image comprises using a rescaled block iterative system.

22. The method of claim 10, wherein reconstructing an image comprises using a maximum-likelihood expectation maximization (MLEM) system.

23. An imaging system comprising:
a nuclear imaging system configured to image an object within an individual;
a respiratory sensor configured to generate a respiratory signal based on respiration of the individual; and
a processor configured to:
define a reference respiratory state for the individual;
obtain reference data representing the reference respiratory state;
obtain, from the respiratory signal, data representing a plurality of respiratory states that together correspond to at least a portion of a cycle of respiration of the individual;
compare data for each of the plurality of respiratory states to a subset of the reference data to obtain a motion estimate of each of the plurality of respiratory states;
compensate for respiratory motion in each of the plurality of respiratory states based on the motion estimates to obtain motion-compensated respiratory state data;
construct an image using the motion-compensated respiratory state data; and
provide an output representative of the image.

24. The system of claim 23, wherein the nuclear imaging system comprises a Single Photon Emission Computed Tomography (SPECT) imaging system.

25. The system of claim 23, wherein the respiratory sensor comprises an air filled girdle or pneumatic bellows.

26. A computer-readable medium storing a computer program for compensating for respiratory motion of an individual in nuclear imaging of an object, the computer program comprising instructions for causing a computer system to:
define a reference respiratory state for the individual;
obtain reference data representing the reference respiratory state;
obtain data representing a plurality of respiratory states that together correspond to at least a portion of a cycle of respiration of the individual;
compare data for each of the plurality of respiratory states to a subset of the reference data to obtain a motion estimate of each of the plurality of respiratory states;
compensate for respiratory motion in each of the plurality of respiratory states based on the motion estimates to obtain motion-compensated respiratory state data;
construct an image using the motion-compensated respiratory state data; and
provide an output representative of the image.

27. A computer-readable medium storing a computer program for compensating for respiratory motion of an individual in nuclear imaging of an object, the computer program comprising instructions for causing a computer system to:
receive projection data associated with imaging of the object at a plurality of discrete projection angles;
receive respiratory signal data associated with respiration of the individual during the imaging;
associate the projection data at the discrete projection angles with a plurality of respiratory states of the individual to obtain amplitude binned respiratory state data for each of the plurality of respiratory states, wherein the plurality of respiratory states are defined based on amplitude levels of the respiratory signal data;
select a reference respiratory state from the amplitude binned data, the reference respiratory state comprising projection data at a first set of discrete projection angles;
select at least one given respiratory state from the amplitude binned data, the at least one given respiratory state comprising projection data at a second set of discrete projection angles;
determine a set of common projection angles from the first and second sets; and
determine a relative position of the object in the given and reference respiratory states based on comparing three-dimensional reconstructions of the object from each of the given and reference respiratory states, wherein each reconstruction is based on projection data at the common projection angles.
